(19) United States
(12) Patent Application Publication
(10) Pub. No.: US 2003/0215866 A1
(43) Pub. Date: Nov. 20, 2003

(54) Models of genetic interactions and methods of use

(76) Inventors: Larry S. Liebovitch, Boca Raton, FL (US); Viktor K. Jirsa, Delray Beach, FL (US); Lina A. Shehadeh, Coconut Creek, FL (US)

Correspondence Address: Margaret J. McLaren, Ph.D., Esq., Akerman Senterfitt., Suite 400, 222 Lakeview Avenue, West Palm Beach, FL, 33402-3188, US

(21) Appl. No.: 10/427,294

(22) Filed: May 1, 2003

Related U.S. Application Data
(60) Provisional application No. 60/376,963, filed on May 1, 2002.

(51) Int. Cl. C12Q 1/68; G06F 19/00; G01N 33/48; G01N 33/50

(52) U.S. Cl. 435/6; 702/20

(57) ABSTRACT

Models of different patterns of genetic interactions were formulated and used in methods to determine the architecture of genetic interactions from mRNA expression levels measured in microarray experiments. The methods can be used to screen biological systems to identify which systems are candidates for therapeutic intervention. Also provided are machine readable storage and systems for using the disclosed models and methods.

### CROSS-REFERENCE TO RELATED APPLICATIONS

This application claims priority of Provisional Patent Application Serial No. 60/376,963 filed May 1, 2002.

### STATEMENT AS TO FEDERALLY SPONSORED RESEARCH

Not applicable.

### FIELD OF THE INVENTION

The invention relates generally to the fields of molecular biology, statistics, complexity theory, and genomics. More particularly, the invention relates to models of patterns of genetic interactions and methods of measuring mRNA levels on a global, genomic scale.

### BACKGROUND OF THE INVENTION

To investigate the genetic bases of different diseases, researchers have focused on the traditional paradigm of trying to identify each specific gene responsible for each specific physiological or pathological function. However, genes regulate each other in a complex web of interactions that is typical of all living things (Chargaff E, 1975, Ann Rev Biochem 44, 1-18). Many physiological or pathological functions are therefore the result of the interactions of many genes. Traditional approaches have proven to be insufficient for studying systems of complex gene interactions. Such approaches are inherently limited in their ability to adequately process the large amount of data from the new high throughput technologies, such as cDNA and oligonucleotide microarrays, that measure mRNA levels simultaneously expressed by thousands of genes (Schena M, ed., 1999, DNA Microarrays: A Practical Approach, Oxford Univ. Press, NY; Rampal J B, ed., 2001, DNA Arrays: Methods and Protocols, Human Press).

Starting with pioneering work on the lac operon (Jacob F and Monod J, 1961, J Mol Biol 3:318-356), there have been quantitative studies of the interactions among small numbers of genes (see, for example, Hlavacek W S and Savageau M A, 1996, J Mol Biol 255:121-139; Liang S et al., 1998, Pac Symp Biocomput 3:18-29; Gardner T S et al., 2000, Nature 403:339-342; Davidson E H et al., 2002, Science, 295:1669-1678). However, there have been very few analyses of the global patterns of interactions between large numbers of genes in the genome.

A simplified model of the global pattern of genetic interactions, termed the NK model, has been proposed (Kauffman S A, 1993, The Origins of Order, Oxford University Press, NY). This model assumes that there are N genes, each of which regulates k other genes. In this model, if k=0, then each gene has no influence on any other gene. Since in this case a mutation in any one gene does not affect any other gene, such a mutation will have only a small effect on the progeny. At the opposite extreme, if k=N, then each gene influences all other genes, such that a mutation in one gene affects all the genes, and the resulting change is so extreme that the progeny will die. As k increases from 0 to N, there is a sharp change (a phase transition) in mutations, from those that have too little effect to those having too much effect. According to this theory, evolution will adjust k so that it has the value at this transition. From analytical and numerical models it was estimated that this transition occurs at about k=5. Thus, each gene should influence about 5 other genes.

Conventional methods to analyze the large amounts of data generated from microarrays have focused on statistical measures such as correlation or cluster analysis (Eisen M B et al., 1998, Proc Natl Acad Sci USA 95:14863-14868; Michaels G S et al., Pac Symp Biocomput 3:42-53; Raychaudhuri S et al., 2000, Pac Symp Biocomput 12:455-466; Brown MPet al., 2000, Proc Natl Acad Sci USA 97:262-267). Given the complexity of genetic interactions, a need exists for models that incorporate the overall architecture of genetic interactions, and provide insight into which biological systems under study might be most fruitful to pursue.

### SUMMARY

The invention relates to methods for analyzing the architecture of genetic interactions. In methods of the invention, mRNA expression levels produced by different models of genetic interactions are computed, and used to infer patterns of genetic interactions within experimental systems, based on patterns of mRNA levels measured in cDNA or oligonucleotide arrays. Using new computational methods from complexity theory, models of different types of genetic interactions are provided. The statistical properties of the mRNA levels produced by the models were computed and compared to the statistical properties of experimentally measured mRNA levels from microarray experiments. Based on the statistics of the measured mRNA levels, the architecture of genetic interaction that best represented the experimental data was determined from the models. The results demonstrate that statistical information from the observed mRNA levels may be used to infer information about the pattern of genetic interactions. The methods of the invention may be used as a means to screen biological systems to identify those which are particularly amendable to manipulation, making them attractive candidates, for example, for development of therapeutic applications. For instance, it might be predicted that a system showing a high degree of hiererarchical clustering of the pattern of gene regulation (i.e., one in which the output of a few genes controls that of many others) would be easier to target therapeutically than one in which many genes contribute equally to the control of others.

Accordingly, the invention provides a method of identifying patterns of genetic interactions in a test system. The method includes the steps of: a) providing models of different patterns of genetic interactions, including at least a first model and a second model; (b) computing statistical measures of mRNA levels produced by the first model and the second model; c) determining statistical measures of mRNA levels in the test system; (d) comparing the computed statistical measures of step (b) to the statistical measures of the test system of step (c); and (e) assigning a pattern of genetic interactions to the test system according to the comparison of step (d).

In the method, the provided model can be selected from Random Output, Random Input, Small World Homogeneous, Small World Heterogeneous and Small World Symmetrical Input/Output models. The statistical measures used in the above steps (b) and (c) of the method can be specified by probability density functions.

The provided model can be formulated. The model can be formulated utilizing a comparison with measurements of mRNA levels from a test system.

In preferred embodiments of the method, the test system can be an oligonucleotide array or a cDNA array.

Also provided is a method of identifying patterns of genetic interactions in a test system, the method including the steps of: (a) providing a plurality of models of genetic interactions, each model specifying a measure of connectivity among genes and corresponding to a unique probability density function of mRNA levels;(b) determining the probability density function of mRNA levels in a test system; (c) selecting the probability density function of at least one of said models which best approximates the probability density function of said test system; and (d) identifying said test system as having a measure of connectivity predicted by the model determined from step (c).

In this method, selected ones of the plurality of models can correspond to low measures of connectivity among genes. This method can further include selecting a test system as a candidate for therapeutic intervention if the predicted measure of connectivity in the test system is low.

Also provided is a method of identifying patterns of genetic interactions in a test system including the steps of: identifying selected characteristics of probability density functions of mRNA levels as indicators of a low measure of connectivity among genes; calculating the probability density function of mRNA levels in said test system; determining whether the probability density function of the test system has characteristics which indicate a low level of connectivity among genes; and if the probability density function of the test system does include these characteristics, identifying the test system as having a low measure of connectivity among genes.

This method can further include selecting a test system as a candidate for therapeutic intervention if the probability density function of mRNA levels in the test system predicts a low measure of connectivity among genes in the test system.

The invention further provides a machine-readable storage having stored thereon, a computer program having a plurality of code sections, the code sections executable by a machine for causing the machine to perform the steps of: (a) providing models of different patterns of genetic interactions, the models including at least a first model and a second model; (b) computing statistical measures of mRNA levels produced by the first model and the second model; (c) determining statistical measures of mRNA levels in a test system; (d) comparing the computed statistical measures of step (b) to the statistical measures of the test system of step (c); and (e) assigning a pattern of genetic interactions according to the comparison of step (d).

In the machine-readable storage, the provided model can be selected from Random Output, Random Input, Small World Homogeneous, Small World Heterogeneous and Small World Symmetrical Input/Output models. The statistical measures of steps (b) and (c) can be specified by probability density functions. The provided model can be formulated. The model can be formulated utilizing a comparison with measurements of mRNA levels from a test system. The test system can be an oligonucleotide array or a cDNA array.

In yet another aspect, the invention includes a machine-readable storage having stored thereon, a computer program having a plurality of code sections, said code sections executable by a machine for causing the machine to perform the steps of: (a)providing a plurality of models of genetic interactions, each model specifying a measure of connectivity among genes and corresponding to a unique probability density function of mRNA levels; (b) determining the probability density function of mRNA levels in a test system; (c) selecting the probability density function of at least one of said models which best approximates the probability density function of said test system; and (d) identifying said test system as having a measure of connectivity predicted by the model determined from step (c).

In this machine-readable storage, selected ones of the plurality of models can correspond to low measures of connectivity among genes. The machine-readable storage can further comprise selecting a test system as a candidate for therapeutic intervention if the predicted measure of connectivity in the test system is low.

The invention further provides a machine-readable storage having stored thereon, a computer program having a plurality of code sections, said code sections executable by a machine for causing the machine to perform the steps of determining the probability density function of mRNA levels in a test system. The machine-readable storage can further comprise selecting a test system as a candidate for therapeutic intervention if the probability density function of mRNA levels in the test system predicts a low measure of connectivity among genes in the test system.

Another aspect of the invention is a system for identifying patterns of genetic interactions in a test system comprising: means for providing models of different patterns of genetic interactions, the models including at least a first model and a second model; means for computing statistical measures of mRNA levels produced by the first model and the second model; means for determining statistical measures of mRNA levels in a test system; means for comparing the computed statistical measures of step (b) to the statistical measures of the test system of step (c); and means for assigning a pattern of genetic interactions according to the comparison of step (d). In this system, the provided model can be a Random Output, Random Input, Small World Homogeneous, Small World Heterogeneous and Small World Symmetrical Input/Output model. The statistical measures of steps (b) and (c) of the system can be specified by probability density functions. The provided model in the system can be formulated. The model can be formulated utilizing a comparison with measurements of mRNA levels from a test system. The test system used with the system for identifying patterns of genetic interactions can be an oligonucleotide array. The test system can be a cDNA array.

The invention further provides a system for identifying patterns of genetic interactions in a test system comprising: means for providing a plurality of models of genetic interactions, each model specifying a measure of connectivity among genes and corresponding to a unique probability density function of mRNA levels; means for determining the probability density function of mRNA levels in a test system; means for determining the probability density function of at least one of said models which best approximates the probability density function of said test system; and means for identifying said test system as having a measure of connectivity predicted by the model which best approximates the probability density function of said test system.

In this system, selected ones of the plurality of models can correspond to low measures of connectivity among genes. The system can further include selecting a test system as a candidate for therapeutic intervention if the predicted measure of connectivity in the test system is low.

Another embodiment of the system is a system for identifying patterns of genetic interactions in a test system including means for determining the probability density function of mRNA levels in the test system. This system can further comprise selecting a test system as a candidate for therapeutic intervention if the probability density function of mRNA levels in the test system predicts a low measure of connectivity among genes in the test system.

In another aspect, the invention provides a method of identifying patterns of genetic interactions in a test system, the method comprising the steps of: (a) measuring expression levels of mRNA for a group of genes; (b) determining the degree of connectivity of said group of genes from said measuring of step (a); (c) repeating steps (a) and (b); and (d) determining sets of genes with lower connectivity.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is five graphs illustrating connection matrices M(i,j) for models of the invention (A1-D) having N=1000 genes. The nonzero elements are black dots. Illustrated models are: (A1) Random Output; (A2) Random Input; (B) Small World Homogeneous Input; (C) Small World Heterogeneous Input; and (D) Small World Symmetric Input/Output.

FIG. 2 is five plots of Log probability density function (PDF) vs. Log x, computed from five different models (A1, A2, B, C, and D) of the connection matrix M(i,j).

FIG. 3 is four plots of PDFs (Log PDF vs. Log x) of mRNA expression levels determined from experimental data sets using arrayed genes from Drosophila.

### DETAILED DESCRIPTION

The invention relates to the formulation of models of different patterns of genetic interactions and methods of using these models to identify patterns of genetic interactions from mRNA levels measured in experiments using cDNA and oligonucleotide arrays. Defining the different patterns of genetic interactions allows for identification of biological systems that are most likely to be productive for further study, and disease systems that are the best candidates for therapeutic intervention. The pattern of genetic interactions in normal tissues, how that pattern changes in altered states, such as cancer, and how different types of altered states (for example, different types of cancers) alter the pattern in different ways can be determined. Understanding these patterns and changes of patterns in genetic interactions may serve as the basis for screening assays, for example to identify different cancers and provide insight into physiology underlying the metastatic transformation of cells.

### Methods of Identifying Patterns of Genetic Interactions

The invention provides a method of identifying patterns of genetic interactions in a test system. The method includes the steps of: a) providing models of different patterns of genetic interactions, including at least a first model and a second model; (b) computing statistical measures of mRNA levels produced by the first model and the second model; c) determining statistical measures of mRNA levels in the test system; (d) comparing the computed statistical measures of step (b) to the statistical measures of the test system of step (c); and (e) assigning a pattern of genetic interactions in the test system according to the comparison of step (d).

Also provided is a method of identifying patterns of genetic interactions in a test system, including the steps of: (a) providing a plurality of models of genetic interactions, each model specifying a measure of connectivity among genes and corresponding to a unique probability density function of mRNA levels;(b) determining the probability density function of mRNA levels in a test system; (c) determining the probability density function of at least one of the models which best approximates the probability density function of the test system, and (d) identifying the test system as having a measure of connectivity predicted by the model determined from step (c).

In this method, selected ones of the plurality of models can correspond to low measures of connectivity among genes. This method can further include selecting a test system as a candidate for therapeutic intervention if the predicted measure of connectivity in the test system is low.

In yet another aspect, the invention provides a method of identifying patterns of genetic interactions in a test system including the step of determining the probability density function of mRNA levels in the test system. This method can further include selecting a test system as a candidate for therapeutic intervention if the probability density function of mRNA levels in the test system predicts a low measure of connectivity among genes in the test system.

### Models of Patterns of Genetic Interactions

In one aspect, the invention provides novel models of the architecture of genetic interactions, formulated on the basis of computed patterns of genetic interactions using several predicted variable parameters.

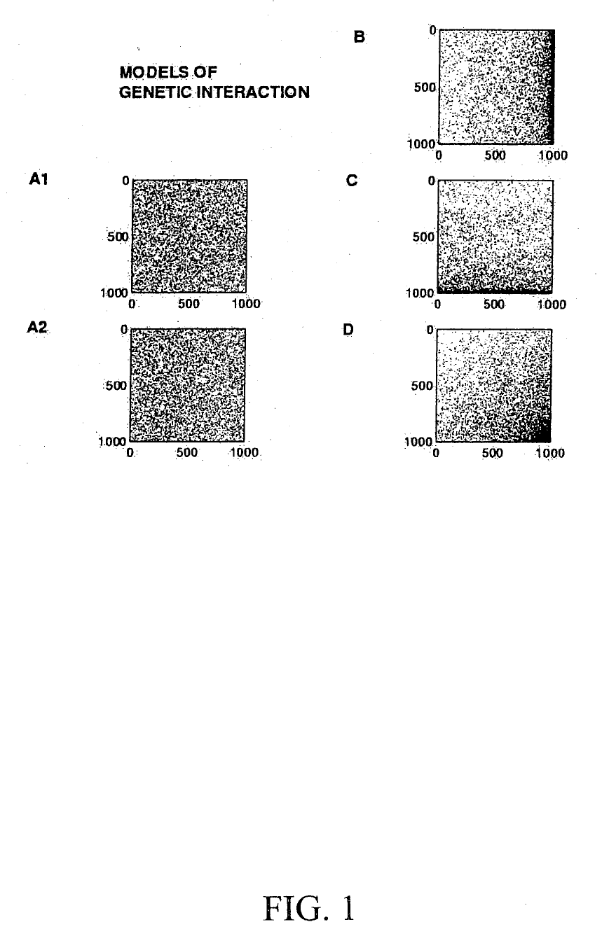

Overview of the models of the invention. The models of the invention provide models of connectivity or “connection matrices,” between genes interacting in a complex multi-gene system. Connection matrices are graphical representations of the levels of gene expression in a system comprised of N genes. Referring to FIG. 1, several connection matrices are illustrated in a system made up of 1000 genes. In the figures, inputs into each gene are represented by each horizontal line. Dots on those lines indicate the genes that have inputs into that specified gene. It is assumed that overall level of gene expression for a given gene is a reflection of the sum of the regulatory inputs (both stimulatory and inhibitory) into that gene from other genes in the system. A low measure of connectivity among genes in a model or test system is indicative of a hierarchical pattern of gene regulation, in which few genes are able to control the expression of many others. Such systems may be selected as candidates for therapeutic intervention.

For a model with N genes, the regulatory input into gene i is represented as an N×N connection matrix M(i,j). The M(i,j) represent the interaction from the j-th gene into the i-th gene. Nonzero values of the M(i,j) are represented by black dots. If the j-th gene has no effect on the i-th gene, then M(i,j)=0. If the j-th gene promotes the expression of the i-th gene, then M(i,j)≠0. The matrix elements of M(i,j) are chosen so that g(k) genes are regulated by k other genes.

Previous models had the same strength of connections in the links between the nodes. (Kauffman S A, 1993, The Origins of Order, Oxford Univ. Press NY; Bower J M and Bolouri H, eds., 2001, Computational Modeling of Genetic and Biochemical Networks, MIT Press, Cambridge Mass.). Models of the invention, by contrast, can account for distributions of connection strengths. Unlike binary models (in which both the nodes and links typically have values that are 0 or 1), the models disclosed herein allow for continuous levels of strengths of interaction and mRNA levels expressed by each gene. Models with other types of g(k) distributions can also be formulated, as well as those with 1) random; and 2) hierarchical connection patterns. Models with different topologies can be formulated, including 1) homogenous models where every gene has the same pattern of input regulation; 2) heterogeneous models where different genes have different patterns of input regulation; and 3) symmetric models where there are similar patterns of input and output regulation.

Five models have been formulated, each with different properties:

Model A1. Random Output. In this model, the output of each gene regulates five other genes chosen at random. The distribution of genetic interactions g(k)=N for k=5, and g(k)=0 for all other k. The connection matrix has 5 values M(i,j)=1 in each column, which were chosen at random. All the other elements M(i,j)=0.

Model A2. Random Input. This model is based on each gene being regulated by the input from five other genes chosen at random. The distribution of genetic interactions g(k)=N for k=5, and g(k)=0 for all other k. The connection matrix has 5 values M(i,j)=1 in each row which were chosen at random. All the other elements M(i,j)=0.

Model B. Small World Homogeneous Input. This model of genetic interactions is patterned after the “small world” form seen in many natural networks. Recent studies of networks have shown that networks of widely diverse types all have a common form (Barabási A L and Albert, R A, 1999, Science 286:509-512; Jeong H et al., 2000, Nature 407:651-654). This commonality has been observed, for example, in such seemingly unrelated networks as the pages on the Internet linked by their URLs, the substations of the electrical power grid of the western United States linked by their transmission lines, actors linked by the number of movies that they appeared in together, and the substrates in metabolic pathways linked by the number of reaction pathways that separate them. The number of nodes g(k) that have k links to other nodes for these networks was found to be a power law; i.e., g(k) is proportional to k\hat{ }(−a). The average distance between nodes in these networks, i.e., the average number of links required to move from any one node to another is surprisingly small. Hence the term “small world” has been coined to describe these networks (Watts D J and Strogatz S H, 1998, Nature 393:440-442). Dense subnetworks of nodes form clusters and a few links bridge one cluster to another. The average distance is small because the links go from the starting node in one dense cluster, to the special links that connect clusters, to the target node. It has been suggested that this architecture is produced when connections in a growing network are added preferentially to nodes that already have the most connections (Albert R and Barabási A L, 2000, Phys Rev Lett 85:5234-5237), or when links are broken and rejoined to other nodes (Huberman B A and Adamic A L, 1999, Nature 401:131-136; Bhan A et al., 2002, Bioinformatics 18:1486-1493).

In the Homogeneous Input version of the Small World model (Model B), the elements of the connection matrix M(i,j) are chosen so that the pattern of inputs into each gene is the same. That is, each gene i has a few inputs from lower order j genes, more inputs from middle order j genes, and many more inputs from higher order j genes. The input from each gene j that regulates gene i has M(i,j)=1 and all the other elements M(i,j)=0. The distribution of the regulatory output from the genes has the “small world” form g(k)=Ak\hat{ }(−a). Model C. Small World Heterogeneous Input. In this model, the elements of the connection matrix M(i,j) were chosen so that the pattern of inputs into each gene is different. Thus as the order of the gene i increases, there are an ever larger number of inputs from the other j genes. The distribution of the regulatory input to the genes has the “small world” form g(k)=Ak\hat{ }(−a). The input from each gene j that regulates gene i has M(i,j)=1 and all the other elements M(i,j)=0. It is noted that Models B and C are related to each other. The outputs from the genes in model B and the inputs to the genes in model C have the same “small world” form of the distribution of genetic interactions g(k). Taking the transform of the matrix M(i,j), i.e., interchanging its rows and columns, reverses the direction of the genetic interactions. Thus, the matrix M(i,j) in model B is the transform of the matrix M(i,j) in model C.

Model D. Small World Symmetric Input/Output. In this model, the distribution of both the regulatory input and output interactions has the “small world” form g(k)=Ak\hat{ }(−a). The input from each gene j that regulates gene i has M(i,j)=1 and all the other elements M(i,j)=0.

In studies described herein, the dependence of the statistical properties of the mRNA levels on the number of genetic interactions in the random models (A1 and A2) and the scaling exponent a in the distribution of genetic interactions g(k)=Ak\hat{ }(−a) of the “small world” models (B, C, and D) was determined.

FIG. 1 illustrates the connection matrices M(i,j) for the five models. The nonzero elements are black dots, each of which is larger than the resolution of one row or column in these figures.

Computing Statistics of mRNA Levels in Models of Genetic Interactions

The connection matrix M(i,j) describes how each gene i is regulated by gene j at any moment in time. Over time, the genes will interact, changing the mRNA level expressed by each gene. Then the genes will interact again, changing the mRNA levels once again. As this process is iterated, the mRNA levels may converge to a steady state. The mRNA level expressed by gene i of the N genes is represented by the column vector X(i). Computation begins with the initial values of the elements of X(i). At each subsequent time step, the new elements of X′(i) are then computed from X′=M·X. This equation is iterated, evolving the mRNA levels in time, until they reach a steady state. The statistical properties of the elements of X(i) are then determined.

An example of a useful statistical property is the probability density function (PDF), i.e., the probability that any of the mRNA levels, x, is between x and x+dx. The PDF is useful for characterizing the global, statistical pattern of the mRNA expression levels. The form of the PDF of all of the mRNA levels reveals the global pattern of the system, without regard to the identity or contributions of individual genes.

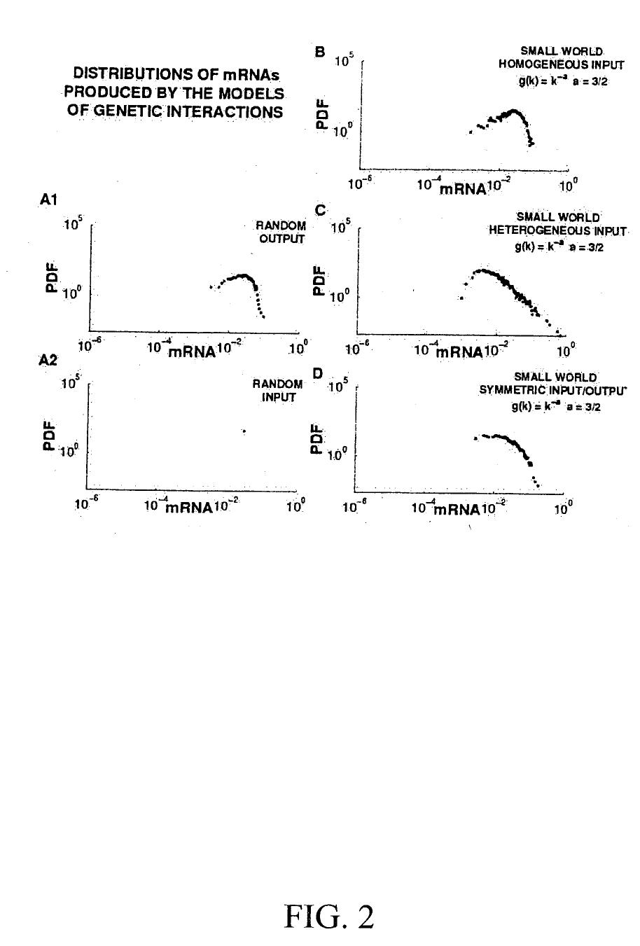

The plots of Log PDF vs. Log x computed from the five different models of the connection matrix M(i,j) are shown in FIG. 2. As can be appreciated from this figure, the functional form of the PDF of the mRNA expression levels differs for each model. The particular characteristics of the PDF curves, such as the width of the curve and the slope of the right hand side of the curve, have been correlated to gene connectivity. In particular, narrower curve widths are correlated with lower measures of connectivity among the genes in the network. Narrower curves indicate a more hierarchical arrangement within the network, i.e., with fewer genes controlling expression of the others. Similarly, steeper slopes on the right hand side of the PDF curves indicate overall control of gene expression by fewer genes. Systems exhibiting such hierarchical characteristics may be preferred as targets for further study and therapeutic intervention because the system is controlled by fewer genes.

The differences between the PDFs of the three “small world” models are notable. The PDF computed from the Symmetric model is intermediate between the PDFs computed from the Homogeneous and Heterogeneous models. This finding is consistent with the fact that the connection matrix M(i,j) of the Symmetric model is intermediate between the connection matrices of the Homogeneous and Heterogeneous models.

On the Log PDF vs. Log x plots (FIG. 2), the curved lines have the form of a stretched exponential, exp[−Bx\hat{ }(−b)], and the straight lines have the form of a power law, x\hat{ }(−c). Note that as b approaches 0, the stretched exponential form approaches a power law. Thus, the progression of the PDFs from the Homogeneous to the Symmetric to the Heterogeneous model can be characterized as a continuous change in the parameter b. The approximate slope c of the monotonically decreasing tail in the three small world models is proportional to the scaling exponent a in the distribution of genetic interactions g(k)=Ak\hat{ }(−a).

From plots such as those shown in FIG. 2, it is apparent that different models of the connection matrix M(i,j) produce different forms in the PDF of the mRNA expression levels. Thus a direct link is demonstrated between the architecture of the genetic interactions and the global, statistical properties of the mRNA expression levels. The global, statistical information from the observed mRNA levels may therefore be useful for inferring information about the architecture of the genetic interactions in a test system.

Determining Statistical Measures of mRNA Levels in a Test System

In the method of identifying patterns of genetic interactions, the above-described models of genetic interactions are used to make predictions about interactions of multiple genes in a test system, by fitting the statistics of global mRNA levels in the test system into a model having known parameters of genetic interactions. Starting from the statistics of the observed mRNA levels, the goal is to use a model to find the pattern of genetic interactions responsible for the experimental data.

The step of determining the statistical measures of mRNA levels in the test system is carried out using experimental data generated from analysis of a test system having multiple genes represented. Any test system suitable for the analysis of mRNA expression levels of multiple genes can be used. Suitable systems include, but are not limited to, arrays of cDNAs (either macroarrays or microarrays), oligonucleotide microarrays and data derived from serial analysis of gene expression (SAGE). SAGE is less able to sample mRNA at low levels, but more accurate at quantifying the mRNA levels that it can detect. Any suitable method for detection of levels of gene expression can be employed. For example, levels of hybridization to cDNA or oligonucleotide targets affixed to a microarray can be measured by recording fluorescent signals generated upon hybridization of a target sequence with a complementary probe that hybridizes to the target.

As an example, in a gene chip system such as that provided by Affymetrix (Santa Clara, Calif.), mRNA levels are measured for each gene by comparing the response of 20 pairs of 25 base probes each with 1) a Perfect Match (PM) probe to a segment of that gene, and 2) a MisMatch (MM) probe, which is a PM probe with one different base in the middle. Such comparisons have been termed “AvgDiff values.” Ideally, the mRNA level would be simply (PM−MM) and would be the same for each probe pair. In reality, however, the (PM−MM) may differ, sometimes markedly for each probe pair and is even sometimes negative. Different algorithms can be used to determine the mRNA levels from the set of 20 PM and MM probes. For example, the largest (PM−MM) value can be chosen, or excluded, or the largest and smallest 25% (PM−MM) values can be excluded and the remaining 50% averaged. The resultant dynamic range of mRNA levels, which is between 100 and 10,000, is sufficient for the PDF analysis.

At the chemical level, nonlinearities in the hybridization reaction resulting from diffusion and concentration dependence can mean that the final fluorescence levels do not fully reflect mRNA abundance. The fluorescent measurements are also limited by the maximum response of the detection system and its degree of linearity. Both of these effects can vary from chip to chip. To overcome this variability, a quantile to quantile normalization procedure can be used to quantitatively compare the results from different chips. Statistical methods can be employed, such as Principle Component Analysis (PCA), and cross-validated prediction using nearest neighbor classification based on differentially expressed genes, to arrive at a PCA cDNA chip molecular classification (see, for example, Giordano T J et al., Am J Pathol 2001, 159:1231-1238).

Any test system capable of measuring mRNA expression under one condition, or of demonstrating changes in mRNA expression under at least two conditions can be used to generate experimental data. For instance, such data can be obtained from experiments using cDNA or oligonucleotide arrays to detect changes in gene expression under particular conditions, such as under varying physiological conditions (for example, light or dark), at different stages of progression of a disease, or, for example, in different types of tumors. A preferred test system for analysis of changes in gene expression in humans is-an array of human genes, for example, in the form of sets of oligonucleotides (“gene chips”) representing thousands of expressed genes.

In studies using the models of the invention, test systems used for comparison with the models included cDNA microarrays containing virtually every gene expressed by yeast cells (Saccharomyces cerevisiae), used to investigate changes in gene expression in yeast cells, both undergoing a metabolic shift, i.e., diauxic shift from anaerobic to aerobic respiration (DeRisi J L et al., Science 278:680-686, 1997), or during synchronized progression through the cell cycle (Spellman P T et al., Mol Biol Cell 9:273-3279, 1998). In another example, the test system was a set of oligonucleotide microarrays containing virtually every gene in the heads of fruit flies (Drosophila). Changes in gene expression were compared in control strains and period null mutants having defective circadian time-keeping (Lin, Y et al., Proc Natl Acad Sci USA 99:9562-9567, 2002). Another test system utilized gene chips from humans (Affymetrix, Santa Clara, Calif.) to analyze changes in gene expression in tumors of different types and stages (Giordano T J et al., Am J Pathol 2001, 159:1231-1238; Beer, D G et al., Nat Med 2002, 8:816-24; Schwartz D R et al., Cancer Res 2002, 62:4722-4729). Further details of methods used for determining statistical measures of mRNA levels in test systems are described in examples below.

Comparing Computed and Experimental Statistical Measures of mRNA Expression

The method of identifying patterns of genetic interactions of the invention includes the step (d) of comparing the computed statistical measures from the models (generated in step (b)) with the statistical measures of mRNA expression calculated from the test system, in order to assign a pattern of genetic interactions.

Such comparisons involve several steps. The various models of the global, statistical measures of the mRNA expression levels are compared to those measured from the experimental data. First, the qualitative form of the PDF and other global, statistical measures of each model are compared to the data. Also, goodness of fit of each model to the experimental data is tested, for example, using a chi-square test. If the PDF of a model seems close to a particular form, such as a Gaussian, Poisson, binomial, hypergeometric, exponential, Weibull, or power law distribution, the goodness of fit of that form to the experimental data is tested. The residuals of the fit are also plotted, to determine if there are systematic deviations between the model and the experimental data. Also determined is how the goodness of fit of each type of model depends on the parameters of that model, and whether linear combinations of models provide a better fit than any one model.

The global, statistical properties of the experimental data may look intermediate or more extreme than any of the initial models. Thus, the comparisons between the initial models and the experimental data may suggest revised parameters of the initial models, or additional types of models. Based on such observations, revised or additional models can be formulated, and the procedures described above can be used to compare the new models to the experimental data. This iterative procedure can thus be used to identify the best models of genetic interactions responsible for the mRNA expression levels found in the experimental data.

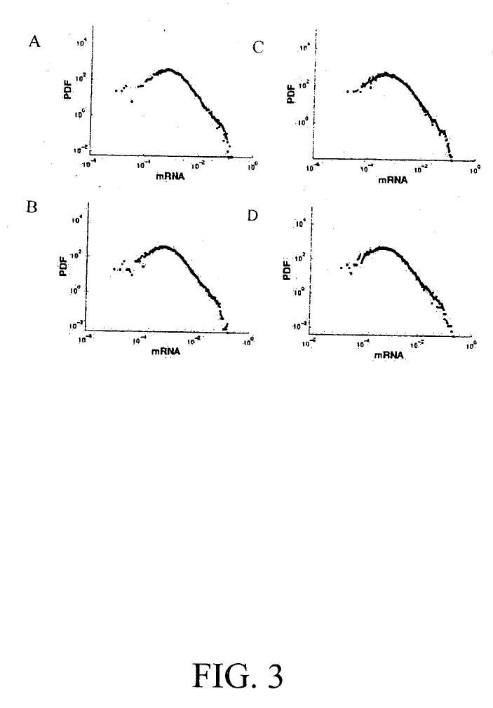

In an example described herein, statistical measures of mRNA expression were computed for a microarray test system including all of the genes of the head of the fruit fly Drosophila, used to analyze changes in gene expression during night and day, both in normal flies and in period mutants having a defect affecting circadian rhythmicity. The statistical measures computed from the experimental data were then compared with the statistical measures of the five models of genetic interaction of the invention. Results of this comparison showed that the experimental data best fit the heterogeneous “small world” model (FIG. 3). These results thus demonstrate that by knowing only the statistics of the mRNA levels (such as PDF), it is possible to determine which of several different architectures of genetic interaction best represents the experimental data. Therefore, global, statistical information from the observed mRNA levels can be used to infer information about the pattern of genetic interactions. Further, this can be accomplished without knowing which gene is responsible for which mRNA level.

By analyzing models with different average numbers of regulatory interactions between genes, it has been found that as the average number of connections increases, the width of the PDF decreases. This may be explained by the fact that as genes become more connected to each other, the mRNA levels of all the genes will approach a single, average value. Thus, the width of the PDF is a direct measure of the average number of interactions between genes. This point illustrates that even very simple aspects of the PDF analysis make it possible to determine important properties of the network of genetic regulation.

### EXAMPLES

The present invention is further illustrated by the following specific examples. The examples are provided for illustration only and are not to be construed as limiting the scope or content of the invention in any way.

### Example 1

### Models of Genetic Interactions

Connection matrix M(i,j). For a model with N genes, the interaction between the genes is represented as an N×N connection matrix M(i,j). If the j-th gene has no effect on the i-th gene then M(i,j)-=0. If the j-th gene regulates the expression of the i-th gene, then M(i,j)≠0.

Issues in Formulating M(i,j). An N×N connection matrix M(i,j) means a restriction to pairwise interactions between the genes. Any three way, or higher order, interactions must be reducible to sums of pairwise interactions. Positive elements of M(i,j) represent stimulatory regulation and negative elements of M(i,j) represent inhibitory regulation. At each subsequent time step the mRNA expression levels of each gene X′(i) are computed from X′=M·X. In some applications, the regulatory elements are limited to only stimulatory interactions so that each element M(i,j)≧0. The reason for this is that when the elements M(i,j)≧0, this is a linear problem and X(i) must converge to a steady state. In other applications, more complex dynamics (which could include limit cycles and chaos) when inhibitory regulation with elements M(i,j)<0 can be included.

Constraints on M(i,j)-Markov model. If X(i) is the fraction of the total mRNA expressed by each gene, and M(i,j) is the probability that the level of mRNA of gene j becomes the level of mRNA of gene i at the next time step, then this problem becomes a standard Markov probability problem (Papoulis A, 1984, Probability, Random Variables, and Stochastic Processes, 2nd Ed., McGraw Hill, N.Y.). Since the sum of the probabilities of being in state i must be equal to 1, the sum of X(i) is 1. Since M(i,j) is a transition probability, M(i,j) must be in the range [0, 1]. Since the sum of the probabilities of all the routes of leaving state j must also be equal to 1, the sum over i of M(i,j)=1. In other words, the sum of the elements M(i,j) in each column is equal to 1. The advantage of this formulation is that Markov models have well known analytical properties that can aid in the computation and that can be used to understand how the steady state mRNA levels depend on M(i,j).

For example, this formulation guarantees that the mRNA levels are normalized (sum X(i)=1) and so do not grow without bound with time. On the other hand, there are also striking disadvantages to this Markov formulation. First, since all M(i,j)≧0, it cannot model inhibitory regulation. Second, the constraints on M(i,j) mean that it is not possible to independently set 1) the strengths of the interactions between the genes and 2) the distribution of genetic interactions that g(k) genes interact with k other genes. For example, since the sum of the elements in each column must be equal to 1, if there are m(j) equal, nonzero elements of M(i,j) in column j, then each element M(i,j)=1/m(j). If there are different numbers of nonzero elements in different columns, then 1/m(j) will be different in each column j and so the input from each gene cannot have the same strength.

Constraints on M(i,j)-Non-Markov model. The inability of the Markov model to represent arbitrary genetic interactions leads to the use of a non-Markov formulation for M(i,j). Hence the more general name of “connection matrix” is used to describe M(i,j) rather than the more restricted “transition probability matrix” used to describe only Markov models. There are no constraints on the choice of the elements M(i,j) in this formulation. The elements M(i,j) are chosen to represent different distributions of genetic interactions g(k), the number of genes g(k) that interact with k other genes. Different connection matrices M(i,j) can have the same distribution of genetic interactions g(k). The advantage of the non-Markov formulation is the ability to develop much more complex models of genetic interactions. The disadvantage is that the mRNA expression levels may grow or decay exponentially as they are evolved in time. This disadvantage is overcome by re-normalizing the mRNA levels (norm X(i)=1) at each time step. Since some models (such as the Random model described below) can be formulated in both the Markov and non-Markov formulation, this gives a direct check on the computational methods used.

Different g(k). Different distributions of genetic interactions g(k), the number of genes g(k) that interact with k other genes, can be examined. These include: 1) single point distributions g(k)=N for k=m and g(k)=0 for k≠m, i.e., each gene is regulated by exactly m other genes; 2) uniform distributions g(k)=B; 3) exponential distributions g(k)=Cexp(−ck); 4) Gaussian distributions g(k)=Eexp[(−k/e)\hat{ }2]; and 5) the “small world” power law distributions g(k)=Ak\hat{ }(−a).

Different Regulatory Strengths. The strength of regulation into gene i from gene j is proportional to the element M(i,j). First, models where regulatory influences all have the same strength, namely, all the nonzero elements of M(i,j)=1 can be examined. Then, models where the regulatory input into gene i is equal in strength from each of m(i) genes can be examined. This means that genes that have a larger number of other genes regulating them will have a smaller input from each gene, namely, M(i,j)=1/m(i). Next, different distributions of strengths chosen at random from uniform, exponential, Gaussian, and power law distributions can be incorporated into the models.

Determining the elements M(i,j) for different g(k). The elements of the connection matrix M(i,j) determine the distribution of genetic interactions g(k). Elements of M(i,j) were chosen to generate a specific g(k). How this will be done is different for each g(k) distribution. As an example, the procedures to be used are illustrated by explaining how this is done for the Random and Small World models.

Random Models. In these models, each gene is equally regulated by exactly m other genes, or equally regulated by a random number of other genes, or regulated at a random strength by a random number of other genes. In the simplest model, each gene is regulated by exactly m other genes. The distribution of genetic interactions g(k) is the single point distribution g(k)=N for k=m and g(k)=0 for k≠m. In each row i of the connection matrix M(i,j), m elements M(i,j)=1 are randomly chosen. All the other M(i,j)=0. In the second model, each gene i is regulated by a number of genes m(i) chosen from a uniform, exponential, Gaussian, or power law distribution. In each row i, m(i) elements are set M(i,j)=1/m(i). All the other M(i,j)=0. In the third model, a random number of elements m(i) in row i are chosen and then those nonzero elements are set M(i,j)=z(k), where the regulatory strength z(k), k=1, 2, . . . , m(i), is chosen at random from a uniform, exponential, Gaussian, or power law distribution.

Small World models. In these models, a few genes interact with many other genes, some genes interact with an intermediate number of other genes, and most genes interact only with a few other genes. The distribution of regulatory input or output has the small world power law form g(k)=Ak\hat{ }(−a). Many different connection matrices M(i,j) can have the same g(k). In homogeneous input models, the regulatory inputs into each gene have the same pattern (the distribution g(k) of the regulatory outputs from the genes have the small world form). In heterogeneous input models, the regulatory inputs into different genes have different patterns (the distribution g(k) of the regulatory inputs into the genes have the small world form). In symmetric models, both the input and output regulations have the same patterns (of the small world form).

For example, the elements of the connection matrix M(i,j) can be determined for a Small World Heterogeneous Input model of N=2000 genes where g(k)=1000k\hat{ }(−1). A simple, albeit crude way to do this is to formulate M(i,j) in discrete blocks by first setting, at random, one element M(i,j)=1 in each of the first 1000 rows of M(i,j). Next, set at random two elements of M(i,j)=1 in each of the next 500 rows of M(i,j). Then, set at random four elements of M(i,j)=1 in each of the next 250 rows of M(i,j). Continue to set at random 2\hat{ }L elements of M(i,j)=1 in each of the next approximately (1000)2\hat{ }(−L) rows. All the other M(i,j)=0.

A second, more sophisticated method can be used to produce a smoother distribution of nonzero elements in M(i,j). In the first method, the L-th block ends at row r=1000[1+1/2+1/4+. . . +1/2\hat{ }(L−1)]=2000[1−2\hat{ }(−L)], and at that row the number of nonzero elements in the (L+1)-th row becomes M=2\hat{ }(L). The number of nonzero elements m in row r is found by substituting m=2\hat{ }(L) into r=2000[1−2\hat{ }(−L)], namely r=2000[1−1/m] and solving for m. Thus, set approximately m=2000/(2000−r) elements of M(i,j)=1 in row r. All the other M(i,j)=0. This relationship can also be generalized to compute m(r) for any value of the scaling exponent a. The number of genetic connections g(k)dk over the interval [k,k+dk] is equal to the number of nonzero elements m(r)dr over the corresponding interval [r,r+dr]. Using the relationship g(k)dk=m(r)dr, it is straightforward to show that m(r)={A/[a(N−r)]}\hat{ }(1/a) generates g(k)=Ak\hat{ }(−a). When the number of elements m in each row computed from these relationships is less than 1, each element is set M(i,j)=1 with probability m/N. That is, a random number q from a uniform distribution is chosen [0,1] and if q<m/N, M(i,j)=1.

Analogous procedures can be used to set the nonzero elements in the columns of M(i,j) for the Small World Homogeneous Input model. The elements of the symmetric model are synmetrical with respect to the diagonal of M(i,j) and that equation for m now determines the density of nonzero elements along lines perpendicular to the diagonal as a function of the distance r along the diagonal from M(1,1) to those perpendicular lines. The approximate number of nonzero elements in those perpendicular lines is that density multiplied by the length of that perpendicular line.

Different organizations of genetic interactions. The procedures described to determine the elements of M(i,j) for a distribution of genetic interactions g(k) determine the values of the m(i) nonzero elements in each row i of the connection matrix M(i,j), but not which elements are the nonzero ones. Therefore, different connection matrices M(i,j) can have the same distribution of genetic interactions g(k). The organization of the nonzero elements in M(i,j) has additional information about the genetic interactions.

### Example 2

### Computation of mRNA Expression

The mRNA expressed by the i-th gene is represented as the i-th element in the column vector X(i). The mRNA expressed by each gene is computed as a function of time from the different models of genetic interaction. Initial values of the mRNA levels are used. At each subsequent time step the new mRNA expression levels X′=M·X. This equation is iterated, evolving the mRNA levels in time, to determine their dynamical behavior and their limiting values if they approach a steady state.

Markov vs. Non-Markov Models. The mRNA levels from Markov models remain bounded since one property of the Markov formulation is that the sum X(i)=1. However, the mRNA levels of the non-Markov models may grow without bound. Therefore, for the non-Markov models the mRNA levels are normalized at each time step by dividing each element in X(i) by the norm (X). The biological interpretation of this procedure is that the elements of X(i) represent the fraction of mRNA expressed by each gene and that the total mRNA expression is limited by biological constraints. Since some models can be formulated as either a Markov or non-Markov model, they can be used to test this re-normalization procedure. For example, the random model where each gene is equally regulated by m other genes can be represented either as a Markov model with all nonzero elements M(i,j)=1/m or as a non-Markov model with the same nonzero elements M(i,j)=1. Whether the re-normalization of the non-Markov model produces a similar statistical pattern of mRNA levels as its equivalent Markov model can be determined. Steady State Solutions. The mRNA expression levels X(i) of some models will converge over time to a steady state. For example, the mRNA levels of the Markov model must reach a steady state. In these Markov models, the property that the sum of the elements M(i,j) in each column is equal to 1 means that the connection matrix M(i,j) has an eigenvalue of 1 and so its corresponding eigenvector 1·X′=M·X′, which means that the mRNA expression levels X′(i) remain the same as they are evolved in time. In any model that reaches a steady state, the mRNA expression levels are the elements of the eigenvector X(i) corresponding to the largest eigenvalue of the connection matrix M(i,j). The steady state mRNA expression levels can therefore be computed in two different ways, i.e., either by iterating X′=M·X until X(i) reaches a steady state, or by finding the eigenvector that corresponds to the largest eigenvalue of the connection matrix M(i,j). The validity of the calculation procedures is confirmed by checking that both methods give the same mRNA expression levels.

Dynamics. The mRNA expression levels X(i) of models that eventually converge to a steady state will first go through a transient period. How the duration and fluctuations of the transient period depend on the type of model, the parameters of each model, and the initial values of X(i) can be determined. The mRNA expression levels X(i) of other models will not converge to a steady state. This is more likely when there is both stimulatory and inhibitory genetic regulation, that is, when the connection matrix has elements of both M(i,j)>0 and M(i,j)<0. The dynamical behavior of these models can be determined, that is, whether the mRNA expression levels reach a limit cycle, chaos, or intermittency. The dynamics are characterized by linear measures such as power spectrum and wavelets, and by nonlinear measures such as the dimensionality of the trajectory in phase space (a chaos measure of the number of degrees of freedom) and the Hurst resealed range analysis (a fractal measure of the range of the fluctuations as a function of the time window).

Analytical relationships between models. The connection matrices M(i,j) of some models are analytically related to the connection matrices of other models. For example, M(i,j) of the Small World Heterogeneous Input model is the transform (interchanging the rows and columns) of M(i,j) of the Small World Homogeneous Input model. Since M(i,j) of these models is not symmetric, that is, the transform of M(i,j) is not equal to the original M(i,j), this means that the eigenvectors and thus the mRNA expression levels of these two models must be different. Such analytical relationships between models can be exploited to better understand how transforming the properties of the connection matrix M(i,j) transforms the statistical properties of the mRNA expression levels.

Statistical analysis of mRNA levels. The global, statistical properties of the mRNA expression levels X(i) are characterized using measures that are independent of which specific gene provides which specific mRNA contribution. The main measure is the probability density function (PDF), i.e., the probability the mRNA levels x are between x and x+dx. The standard method is to compute the PDF from the histogram of the number of values n(x) in each interval [x, Δx], namely PDF(x)=n(x)/(Δ×N). Using this method, when Δx is small, the PDF is well characterized at small x, but poorly characterized at large x, since there are few values in each narrow bin in the tail of the distribution. When Δx is large, more values are captured in each bin at large x, but now the resolution of the PDF is poor at small x. These limitations have been overcome by developing an algorithm that combines histograms of different bins size Δx so that it generates PDFs that are accurate both at small x and at large x (Liebovitch L S et al., 1999, Phys Rev E 59:3312-3319).

Both the standard histogram and a multiple bin size method can be used to determine the PDF of the mRNA expression levels. These PDFs are plotted on linear, semi-logarithmic, and fully logarithmic plots to best determine the functional form of the PDF. The parameters and goodness of fit (using chi-square) of the most likely probability functions expected, i.e., the Gaussian, Poisson, binomial, hypergeometric, exponential, Weibull, and power law distributions, are determined. The effectiveness of other statistical measures, applied to the mRNA expression levels as if the elements in X(i) are a time series, can be evaluated. Since specific genes with specific elements in X(i) are not being identified, the ordering of the elements of X(i) is arbitrary. Therefore, these measures are determined and compared from the original ordering of the elements of X(i), a random shuffling of those elements, and those elements in rank order of their mRNA expression levels. Traditional statistical measures of the moments of the PDF, auotcorrelation function, power spectrum, and principal component analysis can be used, as well as nontraditional measures (Liebovitch L S, 1998, Fractals and Chaos Simplified for the Life Sciences, Oxford Univ Press, NY), such as the phase space dimension from nonlinear dynamics (chaos) and the Hurst exponent from fractals.

Computations. The calculations can be performed, for example, in MATLAB because it is efficient in matrix operations and has good graphical output. Small N×N connection matrices M(i,j) with N=2 and N=3 can be used first, where the numerical output of the computer programs can be verified by analytical calculations. N can then be increased in logarithmic steps, i.e., N=100, 1000, 10,000, 100,000. How the dynamics and the steady state depend on N can be determined. If the results do not depend strongly on N, then the smaller and faster models can be used to more efficiently explore the parameter space. However, if there is a stronger dependence on N, then the parameters will vary more coarsely over a smaller set of slower computations at larger N. The initial values of the mRNA expression levels, the elements of X(i), are either set equal to 0.5, 1, 1/N (so that the sum of the sum X(i)=1), norm(X)/N (so that the norm X(i)=1), or chosen from a uniform distribution over [0,1]. If the mRNA expression levels of a model approach a steady state or a limit cycle, the rate of convergence to that state and its dependence on the initial conditions can be determined. If the dynamics of the mRNA expression levels are chaotic, the Liapunov exponent of those dynamics is determined. The goal of these computations is to construct a dictionary of the global statistical measures of the mRNA expression levels generated by the models with all different combinations of properties. Then the experimental data of mRNA expression level can be compared to the entries in the dictionary, to find the corresponding pattern of genetic interactions.

Computation A: Existence of stationary eigenvectors. The condition for stationarity of an eigenvector is that the corresponding eigenvalue of the connection matrix M is λ=1, where

λX′=MX′ (A1)

means that X′=MX′. If the elements of each column in the matrix sum to 1, then the matrix M can be written as

$$M = ( \begin{matrix}{M}_{11} & {M}_{12} & … & {M}_{1 n} \\ \begin{matrix}{M}_{21} \\ …\end{matrix} & \begin{matrix}… \\ …\end{matrix} & \begin{matrix}… \\ …\end{matrix} & \begin{matrix}… \\ …\end{matrix} \\ 1 - {\sum }_{i = 1}^{n - 1} {M}_{i1} & … & … & 1 - {\sum }_{i = 1}^{n - 1} {M}_{i n}\end{matrix} ) \qquad ( A2 )$$

Adding the first (n−1) rows to the last row, the determinant reads

$$det M = det ( \begin{matrix}{M}_{11} & {M}_{12} & … & {M}_{n} \\ \begin{matrix}{M}_{21} \\ …\end{matrix} & \begin{matrix}… \\ …\end{matrix} & \begin{matrix}… \\ …\end{matrix} & \begin{matrix}… \\ …\end{matrix} \\ 1 & 1 & 1 & 1\end{matrix} ) \qquad \text{(A3)}$$

and then subtracting the first column from all other columns results in

$$\begin{matrix}det M = det ( \begin{matrix}{M}_{11} & {M}_{12} - {M}_{11} & … & {M}_{1 n} - {M}_{11} \\ \begin{matrix}{M}_{21} \\ …\end{matrix} & \begin{matrix}{M}_{22} - {M}_{21} \\ …\end{matrix} & \begin{matrix}… \\ …\end{matrix} & \begin{matrix}{M}_{2 n} - {M}_{21} \\ …\end{matrix} \\ 1 & 0 & 0 & 0\end{matrix} ) = 1 * det B \\ with & \text{(A4)} \\ B = ( \begin{matrix}{M}_{12} - {M}_{11} & … & {M}_{1 n} - {M}_{11} \\ \begin{matrix}{M}_{22} - {M}_{11} \\ …\end{matrix} & \begin{matrix}… \\ …\end{matrix} & \begin{matrix}{M}_{2 n} - {M}_{21} \\ {M}_{( n - 1 ) n} - {M}_{( n - 1 ) 1}\end{matrix}\end{matrix} ) & \text{(A5)}\end{matrix}$$

The determinants of the matrices M and B may be written as the product of the eigenvalues λ_{Mi}, i=1, . . . n and λ_{Bi}, i=1, . . . n−1, respectively, that is

λ_{M1}λ_{M2} . . . λ_{Mn}=1 * λ_{B1}λ_{B2} . . . λ_{B(n-1)} (A6)

From this, it follows that

$${λ}_{M1} = \frac{{γ}_{B1} {γ}_{B2} {…γ}_{B ( n - 1 )}}{{γ}_{M2} {…γ}_{Mn}} = 1 \qquad \text{(A7)}$$

because

$$detB = det ( \begin{matrix}{M}_{12} & … & {M}_{1 n} \\ \begin{matrix}{M}_{22} \\ …\end{matrix} & \begin{matrix}… \\ …\end{matrix} & \begin{matrix}{M}_{2 n} \\ {M}_{( n - 1 ) n}\end{matrix}\end{matrix} ) - det ( \begin{matrix}{M}_{11} & … & {M}_{11} \\ \begin{matrix}{M}_{21} \\ …\end{matrix} & \begin{matrix}… \\ …\end{matrix} & \begin{matrix}{M}_{21} \\ {M}_{( n - 1 ) 1}\end{matrix}\end{matrix} ) \qquad \text{(A8)}$$

The second determinant is 0 since its columns are identical. From this point on, we will refer to the stationary eigenvalue as λ₀=1.

Hence, if the matrix columns sum to 1, then there is always an eigenvalue λ₀=1 which guarantees the existence of a non-trivial stationary state. The same applies when the matrix rows sum to 1, that is for the transpose M^{T}, since det M=detM^{T}.

Computation B: Convergence to stationary eigenvectors. If an eigenvector is stationary, λ₀=1, all other eigenvectors in the system might or might not converge to it. This convergence will be characterized by the stability of the other eigenvectors and their corresponding eigenvalues λᵢ, for i=1, 2, . . . n. If |λᵢ|>1, the system is unstable. If |λᵢ|<1, the system is stable. Under the condition in which the elements in the matrix columns or rows sum to one, the numerical simulations in this work have shown that |λᵢ|<1, i=1, . . . , n and there will always be a unique solution where λ₀=1. Therefore, the system will converge to the stationary eigenvector.

Computation C: Uniform eigenvectors. The output column vector X′ of the connection matrix M is determined, where X is the input vector and norm(X)=1.

X′=MX (C1)

If each row in the sparse matrix M has m equal nonzero elements that sum to 1, and all others are zero, that is

$${\sum }_{j}^{n} {M}_{ij} = 1 , then the model can be written as , \\ ( \begin{matrix}{X}_{1}^{′} \\ {X}_{2}^{′} \\ ⋯ \\ {X}_{n}^{′}\end{matrix} ) = ( \begin{matrix}0 & \frac{1}{m} & ⋯ & \frac{1}{m} \\ \frac{1}{m} & 0 & \frac{1}{m} & ⋯ \\ ⋯ & \frac{1}{m} & 0 & 0 \\ ⋯ & ⋯ & ⋯ & ⋯\end{matrix} ) ( \begin{matrix}{X}_{1} \\ {X}_{2} \\ ⋯ \\ {X}_{n}\end{matrix} ) \qquad ( C2 )$$

Assuming a uniform initial state vector

$${X}_{u} = ( \begin{matrix}1 \\ 1 \\ ⋯ \\ 1\end{matrix} ) \qquad \text{(C3)}$$

Inserting the uniform state vector in (C2), one obtains for each component

$${X}_{i}^{′} = {\sum }_{n = 1}^{m} \frac{1}{m} = 1 ∀ i \qquad \text{(C4)}$$

and thus the uniform state vector is the unique stationary eigenvector with λ₀=1.

For an arbitrary initial state vector, the following is true: As shown in Computation A and B above, the system will converge to the unique stationary eigenvector, in this case the uniform state vector Xᵤ with the weight

$$\begin{matrix}{X}_{u} = \overline{X} ( \begin{matrix}1 \\ 1 \\ ⋯ \\ 1\end{matrix} ) \\ where & \text{(C5)} \\ \overline{X} = {\sum }_{i} {X}_{i} & \text{(C6)}\end{matrix}$$

Computation D: Derivation of the density function. The regulatory influence at each time step of the j-th gene on the i-th gene is given by the element Mij in the N×N connection matrix M. If g genes regulate k genes, the distribution of genetic interactions is defined by the function g(k). The “small world” networks described herein have the form g(k)=Ak^{-a}, where 1≦a≦6. It is necessary to know how to choose the elements in M to generate the desired g(k). The choice of A is arbitrary. The distribution of mRNA levels, the elements Xᵢ of the 1-D steady state column vector X produced by iterating X′=MX, does not depend on A, since X is renormalized at each time step. A was chosen here so there are 6000 nonzero elements in the N=1000 connection matrices. To produce the desired a it must be determined how to choose the density Y (R) of nonzero elements in each line that is at a distance R from the start of the matrix, M₁₁.

Method 1. The scale free form means that the exponent −a is the slope of In g(k) versus In k. That is, multiplying k by a constant factor, g(k) changes by another constant factor, and the ratio of these two factors determines a. For example, if g(k) is multiplied by 1/λ so that

$$g ( {k}_{2} ) = \frac{1}{γ} g ( {k}_{1} ) \qquad \text{(D1)}$$

and k is multiplied by 2 so that k₂=2k₁, then the slope is

$$- a = \frac{lng ( {k}_{2} ) - lng ( {k}_{1} )}{{lnk}_{2} - {lnk}_{1}} = - \frac{ln λ}{ln 2} \qquad ( D2 )$$

If the nonzero elements of matrix M are chosen in blocks, one could choose one nonzero element in the first b lines, two nonzero elements in the next b/λ lines, four nonzero elements in the next b/λ² lines, and so on. For the homogeneous model, the lines are the rows and R is the number of rows from the top row to the desired row. For the heterogeneous model, the lines are the columns and R is the number of columns from the first column to the desired column. For the symmetric model, the lines are the diagonals and R is the distance from M₁₁ to the midpoint of the desired diagonal. The first diagonal extends from M₁₂ to M₂₁, the next diagonal extends from M₁₃ to M₃₁, and so on. The line at distance R that has Y=2^{h} nonzero elements is at

$$R = {\sum }_{1 = 0}^{h} \frac{b}{{λ}^{h}}$$

Using the standard formula that the sum of a geometric progression, with r=1/λ, namely,

$${\sum }_{i = 0}^{h} {br}^{h} = \frac{b}{1 - r} - \frac{{br}^{h}}{1 - r} \qquad ( D3 )$$

then

$$R = N - N {λ}^{\frac{- lny}{ln2}} \\ \frac{b}{1 - \frac{1}{λ}} \text{and we use the fact that} h = \frac{- lny}{ln2} . \qquad ( D4 )$$

Solving for Y,

$$Y ( R ) = {[ \frac{N}{N - R} ]}^{\frac{1}{a}} \qquad ( D5 )$$

Method 2. A more general relationship between Y (R) and g(k) can be found by noting that the number of genes regulated, g(k)dk, over the interval [k; k+dk] is equal to the number of nonzero elements, Y (R)dR, over the corresponding interval [R;R+dR]. Thus, g(k)dk=Y (R)dR where Y (R)=k. To generate the “small world”models, if the density has the form.

$$Y ( R ) = {[ \frac{A}{a ( N - R )} ]}^{\frac{1}{a}} \qquad ( D6 )$$

then

$$R = N - \frac{A}{a} {k}^{- a} \qquad ( D7 )$$

and therefore, the distribution is

$$g ( k ) = Y ( R ) \frac{ⅆ R}{ⅆ k} = {Ak}^{- a} \qquad ( D8 )$$

### Example 4

### Analysis of Experimental Data from Gene Chips

The models of the invention were used to analyze global mRNA expression patterns available in several databases. The same global, statistical methods that were used to analyze the models were also used to analyze the experimental data. Data sets. cDNA and oligonucleotide microarrays have been previously used to measure the mRNA levels expressed by virtually every gene in the yeast Saccharomyces cerevisiae. For example, mRNA expression in the shift from the anaerobic metabolism of sugar to the aerobic metabolism of ethanol has been measured (DeRisi J L et al., 1997, Science 278:680-686). Expression of mRNA has also been evaluated as yeast progress through the mitotic cell cycle after being arrested in G1 by the alpha pheromone, or late in mitosis by a temperature pulse (Spellman P T et al., 1998, Biol Cell 9:3273-3297). In these databases, only relative mRNA levels are provided, normalized to the values for the initial time points for each gene, and the dynamic range of the mRNA levels is limited in these data sets. Initial studies were performed using these datasets to generate statistical measures of mRNA levels in experimental test systems, for comparison with computed statistical measures produced by the models of the invention.

A data set from Affymetrix (Santa Clara Calif.) was used that is in the form of AvgDiff values (comparisons between PM and MM probes), which are readings from one experimental condition. This is in contrast to previous measurements from spotted chips, which used Cy3 and Cy5dyes to calculate the relative mRNA abundance ratios (DeRisi J L et al., 1997, Science 278:680-686; Spellman PT et al., 1998, Biol Cell 9:3273-3297). In addition, this data had a greater dynamic range than the above-described data sets, having a factor of at least 100 between the highest and lowest mRNA values.

The PDFs were calculated from this study, in which cDNA microarray technology was used to measure daily mRNA expression levels of virtually every gene in the heads of control and period null mutant Drosophila flies (Lin, Y et al., Proc. Natl Acad Sci USA 99:9562-9567, 2002). Period is one of several genes in Drosophila that are necessary for the normal functioning of the circadian time-keeping system. The datasets of the study were distributed among four different experiments. In each experiment, gene expression was measured at 4 hour intervals over a 24 hour period as follows. Experiment A: Control dark-dark cycle. Gene expression was measured from control, wild type flies kept in constant darkness. Two replicate time series were recorded. Experiment B: Control light-dark cycle. Gene expression was measured from control, wild type flies kept in light-dark cycles. Three replicate time series were recorded. Experiment C: period null mutant dark-dark cycle. Gene expression was measured from per mutant flies kept in constant darkness. Two replicate time series were recorded. Experiment D: period null mutant light-dark cycle. Gene expression was measured from per mutant flies kept in light-dark cycles. Two replicate time series were recorded.

Experimental data were in the form of fluorescence measurements, representing average values of fluorescence for each spot on the microarray. (The fluorescence levels are reflective of relative abundance of mRNA species in the test RNA sample, determined from the level of hybridization of fluorescently tagged probe mRNA to the oligonucleotides on the array.) PDFs were calculated from these data sets using the fluorescence values to calculate distribution of mRNA expression levels in the data.

The results showed that the PDFs of the mRNA expression levels from the nine time series were quite similar. Representative plots from the last (sixth) time points of Log PDF vs. Log x from each of the four experiments (A-D) are shown in FIG. 3.

### Example 5

### Comparison of Models and Experimental Data

The PDFs of the experimental data shown in FIG. 3 have a prominent monotonically decreasing tail that is a straight line with an approximate slope of 2 on the plots of Log PDF vs. Log x. By comparison of the plots of the experimental data with the theoretical models of genetic interaction, it was determined that qualitatively, the PDFs of the experimental data were most like the Small World Heterogeneous Input model of genetic interactions. In this model, different genes have different patterns of regulatory input, but all the genes have the same pattern of regulatory output to other genes. That is, some genes are regulated by the input from few other genes, whereas other genes are regulated by the input of many other genes. However, all the genes have a similar pattern of regulatory output onto other genes.

The slope of the experimental data was closest to models where the distribution of genetic interactions, i.e., the number of genes g(k) that are regulated by k genes, is g(k)=Ak\hat{ }(−a), where the value of the scaling exponent a=3/2. Results of this study therefore demonstrate that this model is able to predict global patterns of mRNA expression determined from experimental data.

### Example 6

### Analysis of Statistical Measures of Gene Interactions in Tumors

Data sets. Analysis of cDNA chip data has been used to develop a molecular classification of tumor types. In one study, data were obtained using microarrays consisting of 7129 probe sets, each representing a transcript (HuGeneFL chip, Affymetrix, Santa Clare, Calif.). Gene expression profiles were generated from 154 primary adenocarcinomas (57 lung, 51 colon, and 46 ovary). Differences were identified that allowed discrimination of tumors in an organ-specific manner and revealed genes that are potentially useful as diagnostic markers for these tumors. A second study used oligonucleotide arrays to measure gene expression profiles for 86 primary lung adenocarcinomas, including 67 stage I and 19 stage III tumors, and 10 non-neoplastic lung samples. Using hierarchical clustering on 4,966 genes, three clusters were found that showed significant differences with respect to tumor stage and tumor differentiation (Beer, D G et al., Nat Med 2002, 8:816-24). In a third study, oligonucleotide microarrays were used to generate gene expression profiles for ovarian epithelial tumors, which are morphologically heterogeneous, and are classified histologically under four types of primary adenocarcinomas, i.e., serous, mucinous, endometrioid, and clear cell. Using PCA, different mRNA expression profiles were generated for these major classes of ovarian tumors (Schwartz DR et al., Cancer Res 2002, 62:4722-4729).

Data sets such as those from the above studies can be used to determine if different tumor types, or stages of tumors, have the same, or different, patterns of genetic regulatory networks. The statistical measures of the mRNA levels are determined, for example, by calculating the PDFs. Analysis of error in the experimental data from the test system is first performed to estimate the accuracy and reliability of the system. To provide an estimate of chip errors, PDFs are compared from different cDNA microarray chips used to analyze the mRNA from the same cells. To estimate biological heterogeneity, PDFs are compared from mRNA from different probes of the same tumor. To provide an estimate of variations between individuals, PDFs from samples of similar tumor types and stages are compared. The error analysis thus performed reveals the statistical significance of the relative contributions of observed variations in the PDFs between chips, cells, individuals, tumor types, and stages of tumors.

Having established the sources of experimental and biological variation, differences between tumor types and stages are then determined. Qualitative and quantitative fit between the experimental PDFs and those of the different models of the pattern of genetic regulation are calculated using methods described above. Observed differences in PDFs between tumor types and stages are then correlated with their PCA cDNA chip molecular classification, as well as with established biomedical classifiers such as morphology and immunochemical staining. In this manner, it can be determined how the pattern of network of genetic regulation varies according to the tumor type, stage, and survival probability.

### Other Embodiments

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

The present invention can be implemented using commercially available software development tools, such as Matlab or other suitable data analysis tools. Accordingly, the present invention can be realized in hardware, software, or a combination of hardware and software. The present invention can be realized in a centralized fashion in one computer system or in a distributed fashion where different elements are spread across several interconnected computer systems. Any kind of computer system or other apparatus adapted for carrying out the methods described herein is suited. A typical combination of hardware and software can be a general-purpose computer system with a computer program that, when being loaded and executed, controls the computer system such that it carries out the methods described herein.

The present invention also can be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

This invention can be embodied in other forms without departing from the spirit or essential attributes thereof. Accordingly, reference should be made to the following claims, rather than to the foregoing specification, as indicating the scope of the invention.

What is claimed is:

1. A method of identifying patterns of genetic interactions in a test system, the method comprising the steps of:
(a) providing models of different patterns of genetic interactions, the models including at least a first model and a second model;
(b) computing statistical measures of mRNA levels produced by the first model and the second model;
(c) determining statistical measures of mRNA levels in a test system;
(d) comparing the computed statistical measures of step (b) to the statistical measures of the test system of step (c); and
(e) assigning a pattern of genetic interactions according to the comparison of step (d).

2. The method of claim 1, wherein the provided model is selected from the group consisting of a Random Output, Random Input, Small World Homogeneous, Small World Heterogeneous and Small World Symmetrical Input/Output model.

3. The method of claim 1, wherein the statistical measures of steps (b) and (c) are specified by probability density functions.

4. The method of claim 1, wherein the provided model is formulated.

5. The method of claim 4, wherein the model is formulated utilizing a comparison with measurements of mRNA levels from a test system.

6. The method of claim 1, wherein the test system is an oligonucleotide array.

7. The method of claim 1, wherein the test system is a cDNA array.

8. A method of identifying patterns of genetic interactions in a test system, the method comprising the steps of:
(a) providing a plurality of models of genetic interactions, each model specifying a measure of connectivity among genes and corresponding to a unique probability density function of mRNA levels;
(b) determining the probability density function of mRNA levels in a test system;
(c) selecting the probability density function of at least one of said models which best approximates the probability density function of said test system; and
(d) identifying said test system as having a measure of connectivity predicted by the model determined from step (c).

9. The method of claim 8, wherein selected ones of the plurality of models correspond to low measures of connectivity among genes.

10. The method of claim 9, further comprising selecting a test system as a candidate for therapeutic intervention if the predicted measure of connectivity in the test system is low.

11. A method of identifying patterns of genetic interactions in a test system comprising the steps of:
identifying selected characteristics of probability density functions of mRNA levels as indicators of a low measure of connectivity among genes;
calculating the probability density function of mRNA levels in said test system;
determining whether said probability density function of said test system has characteristics which indicate a low level of connectivity among genes; and
if said probability density function of said test system does include said characteristics, identifying said test system as having a low measure of connectivity among genes.

12. The method of claim 11, further comprising selecting a test system as a candidate for therapeutic intervention if the probability density function of mRNA levels in said test system predicts a low measure of connectivity among genes in the test system.

13. A machine-readable storage having stored thereon, a computer program having a plurality of code sections, said code sections executable by a machine for causing the machine to perform the steps of:
(a) providing models of different patterns of genetic interactions, the models including at least a first model and a second model;
(b) computing statistical measures of mRNA levels produced by the first model and the second model;
(c) determining statistical measures of mRNA levels in a test system;
(d) comparing the computed statistical measures of step (b) to the statistical measures of the test system of step (c); and
(e) assigning a pattern of genetic interactions according to the comparison of step (d).

14. The machine-readable storage of claim 13, wherein the provided model is selected from the group consisting of a Random Output, Random Input, Small World Homogeneous, Small World Heterogeneous and Small World Symmetrical Input/Output model.

15. The machine-readable storage of claim 13, wherein the statistical measures of steps (b) and (c) are specified by probability density functions.

16. The machine-readable storage of claim 13, wherein the provided model is formulated.

17. The machine-readable storage of claim 16, wherein the model is formulated utilizing a comparison with measurements of mRNA levels from a test system.

18. The machine-readable storage of claim 13, wherein the test system is an oligonucleotide array.

19. The machine-readable storage of claim 13, wherein the test system is a cDNA array.

20. A machine-readable storage having stored thereon, a computer program having a plurality of code sections, said code sections executable by a machine for causing the machine to perform the steps of:
(a) providing a plurality of models of genetic interactions, each model specifying a measure of connectivity among genes and corresponding to a unique probability density function of mRNA levels;
(b) determining the probability density function of mRNA levels in a test system;
(c) determining the probability density function of at least one of said models which best approximates the probability density function of said test system; and
(d) identifying said test system as having a measure of connectivity predicted by the model determined from step (c).

21. The machine-readable storage of claim 18, wherein selected ones of the plurality of models correspond to low measures of connectivity among genes.

22. The machine-readable storage of claim 21, further comprising selecting a test system as a candidate for therapeutic intervention if the predicted measure of connectivity in the test system is low.

23. A machine-readable storage having stored thereon, a computer program having a plurality of code sections, said code sections executable by a machine for causing the machine to perform the steps of:
identifying selected characteristics of probability density functions of mRNA levels as indicators of a low measure of connectivity among genes;
calculating the probability density function of mRNA levels in said test system;
determining whether said probability density function of said test system has characteristics which indicate a low level of connectivity among genes; and
if said probability density function of said test system does include said characteristics, identifying said test system as having a low measure of connectivity among genes.

24. The machine-readable storage of claim 23, further comprising selecting a test system as a candidate for therapeutic intervention if the probability density function of mRNA levels in said test system predicts a low measure of connectivity among genes in the test system.

25. A system for identifying patterns of genetic interactions in a test system comprising:
means for providing models of different patterns of genetic interactions, the models including at least a first model and a second model;
means for computing statistical measures of mRNA levels produced by the first model and the second model;
means for determining statistical measures of mRNA levels in a test system;
means for comparing the computed statistical measures of the first and second models to the statistical measures of the test system; and
means for assigning a pattern of genetic interactions according to the comparison of the computed statistical measures of the first and second models and the test system.

26. The system of claim 25, wherein the provided model is selected from the group consisting of a Random Output, Random Input, Small World Homogeneous, Small World Heterogeneous and Small World Symmetrical Input/Output model.

27. The system of claim 25, wherein the statistical measures of steps (b) and (c) are specified by probability density functions.

28. The system of claim 25, wherein the provided model is formulated.

29. The system of claim 25, wherein the model is formulated utilizing a comparison with measurements of mRNA levels from a test system.

30. The system of claim 25, wherein the test system is an oligonucleotide array.

31. The system of claim 25, wherein the test system is a cDNA array.

32. A system for identifying patterns of genetic interactions in a test system comprising:
means for providing a plurality of models of genetic interactions, each model specifying a measure of connectivity among genes and corresponding to a unique probability density function of mRNA levels;
means for determining the probability density function of mRNA levels in a test system;
means for selecting the probability density function of at least one of said models which best approximates the probability density function of said test system; and
means for identifying said test system as having a measure of connectivity predicted by the model which best approximates the probability density function of said test system.

33. The system of claim 32, wherein selected ones of the plurality of models correspond to low measures of connectivity among genes.

34. The system of claim 32, further comprising selecting a test system as a candidate for therapeutic intervention if the predicted measure of connectivity in the test system is low.

35. A system for identifying patterns of genetic interactions in a test system comprising:
means identifying selected characteristics of probability density functions of mRNA levels as indicators of a low measure of connectivity among genes;
means for calculating the probability density function of mRNA levels in said test system;
means for determining whether said probability density function of said test system has characteristics which indicate a low level of connectivity among genes; and
if said probability density function of said test system does include said characteristics, means for identifying said test system as having a low measure of connectivity among genes.

36. The system of claim 35, further comprising selecting a test system as a candidate for therapeutic intervention if the probability density function of mRNA levels in said test system predicts a low measure of connectivity among genes in the test system.

37. The method of claim 11, wherein at least one of the selected characteristics is a width specified by the probability density function curve.

38. The method of claim 11, wherein at least one of the selected characteristics is a slope of the right hand portion of the probability density function curve.

39. The machine-readable storage of claim 23, wherein at least one of the selected characteristics is a width specified by the probability density function curve.

40. The machine-readable storage of claim 23, wherein at least one of the selected characteristics is a slope of the right hand portion of the probability density function curve.

41. The system of claim 35, wherein at least one of the selected characteristics is a is a width specified by the probability density function curve.

42. The system of claim 35, wherein at least one of the selected characteristics is a slope of the right hand portion of the probability density function curve.

43. A method of identifying patterns of genetic interactions in a test system, the method comprising the steps of:
(a) measuring expression levels of niRNA for a group of genes;
(b) determining the degree of connectivity of said group of genes from said measuring of step (a);
(c) repeating steps (a) and (b); and
(d) determining sets of genes with lower connectivity.
